(19) United States
(12) Patent Application Publication
(10) Pub. No.: US 2010/0034890 A1
(43) Pub. Date: Feb. 11, 2010

(54) COMBINATIONS OF FORMOTEROL AND FLUTICASONE PROPIONATE FOR ASTHMA

(76) Inventors: Jeremy Guy Clarke, Bath (GB); Henry Luke Danahay, Horsham (GB); Ian F. Hassan, Morris Plains, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/581,532

(22) Filed: Oct. 19, 2009

Related U.S. Application Data
(63) Continuation of application No. 11/196,560, filed on Aug. 3, 2005, now abandoned.
Application No. 11/196,560 is a continuation of application No. 10/718,209, filed on Nov. 20, 2003, now abandoned.
Application No. 10/718,209 is a continuation of application No. 09/930,337, filed on Aug. 15, 2001, now abandoned.
Application No. 09/930,337 is a continuation of application No. PCT/EP00/01270, filed on Feb. 16, 2000.

(30) Foreign Application Priority Data
Feb. 18, 1999 (GB) 9903759.0

(51) Int. Cl. A61K 9/14; A61K 31/56; A61K 9/12

(52) U.S. Cl. 424/489; 514/171; 424/45

(57) ABSTRACT

A pharmaceutical composition comprising (A) formoterol or a pharmaceutically acceptable salt thereof or a solvate of formoterol or said salt and (B) fluticasone propionate, suitable for use in the treatment of inflammatory or obstructive airways diseases.

This invention relates to combinations of a beta-2 agonist and a steroid and their use for the treatment of inflammatory or obstructive airways diseases.

Formoterol,N-[2-hydroxy-5-(1-hydroxy-2-((2-(4-methoxyphenyl)-1-methylethyl)amino)-ethyl)phenyl]formamide, particularly in the form of its fumarate salt, is a bronchodilator used in the treatment of inflammatory or obstructive airways diseases. Fluticasone propionate, S-fluoromethyl 6α,9α-difluoro-11β-hydroxy-16α-methyl-3-oxo-17α-propionyloxyandrosta-1,4-diene-17β-carbothioate, an anti-inflammatory corticosteroid, is described in U.S. Pat. No. 4,335,121.

It has now surprisingly been found that a significant unexpected therapeutic benefit, particularly a synergistic therapeutic benefit, in the treatment of inflammatory or obstructive airways diseases can be obtained by using a composition containing formoterol, or a salt or solvate thereof, and fluticasone propionate. For instance, it is possible using such a composition to reduce the dosages of fluticasone propionate required for a given therapeutic effect considerably compared with those required using treatment with fluticasone propionate alone, thereby minimising possibly undesirable side effects. In particular, it has been found that compositions containing formoterol and fluticasone propionate induce an anti-inflammatory activity which is significantly greater than that induced by formoterol or fluticasone propionate alone and that the amount of fluticasone propionate needed for a given anti-inflammatory effect may be significantly reduced when used in admixture with formoterol, thereby reducing the risk of undesirable side effects from the repeated exposure to the steroid involved in the treatment of inflammatory or obstructive airways diseases.

Furthermore, using the compositions of the invention, medicaments which have a rapid onset of action and a long duration of action may be prepared. Moreover, using the compositions of the invention, medicaments which result in a significant improvement in lung function may be prepared. In another aspect, using the compositions of the invention, medicaments which provide improved control of obstructive or inflammatory airways diseases, or a reduction in exacerbations of such diseases, may be prepared. In a further aspect, using compositions of the invention, medicaments which can be used on demand in rescue treatment of obstructive or inflammatory airways diseases, or which reduce or eliminate the need for treatment with short-acting rescue medicaments such as salbutamol or terbutaline, may be prepared; thus medicaments based on compositions of the invention facilitate the treatment of an obstructive or inflammatory airways disease with a single medicament.

Accordingly, in one aspect, the present invention provides a pharmaceutical composition comprising (A) formoterol or a pharmaceutically acceptable salt thereof or a solvate of formoterol or said salt and (B) fluticasone propionate.

In another aspect, the present invention provides a method of treating an inflammatory, or obstructive airways disease which comprises administering to a subject in need of such treatment an effective amount of a pharmaceutical composition comprising (A) and (B) as hereinbefore defined.

In a further aspect, the present invention provides a pharmaceutical composition comprising a mixture of effective amounts of (A) and (B) as hereinbefore defined together with a pharmaceutically acceptable carrier.

In a yet further aspect, the present invention provides a pharmaceutical composition for use in the treatment of an inflammatory or obstructive airways disease comprising (A) and (B) as hereinbefore defined.

The present invention still further provides the use of a pharmaceutical composition comprising (A) and (B) as hereinbefore defined for the preparation of a medicament for the treatment of an inflammatory or obstructive airways disease.

Pharmaceutically acceptable salts of formoterol include, for example, salts of inorganic acids such as hydrochloric, hydrobromic, sulfuric and phosphoric acids, and organic acids such as fumaric, maleic, acetic, lactic, citric, tartaric, ascorbic, succinic, glutaric, gluconic, tricarballylic, oleic, benzoic, p-methoxybenzoic, salicylic, o- and p-hydroxybenzoic, p-chlorobenzoic, methanesulfonic, p-toluenesulfonic and 3-hydroxy-2-naphthalene carboxylic acids.

Component (A) may be in any isomeric form or mixture of isomeric forms, foe example a pure enantiomer, a mixture of enantiomers, a racemate or a mixture thereof. It may be in the form of a solvate, for example a hydrate, thereof, for example as described in U.S. Pat. No. 3,994,974 or U.S. Pat. No. 5,684,199, and may be present in a particular crystalline form, for example as described in WO95/05805. Preferably, component (A) is formoterol fumarate, especially in the form of the dihydrate.

Administration of the pharmaceutical composition as hereinbefore described is preferably by inhalation, in which case (A) and (B) are in inhalable form. The inhalable form of the composition may be, for example, an atomizable composition such as an aerosol comprising the active ingredients, i.e. (A) and (B), in solution or dispersion in a propellant, or a nebulizable composition comprising a dispersion of the active ingredients in an aqueous, organic or aqueous/organic medium. For example, the inhalable form of the pharmaceutical composition may be an aerosol comprising a mixture of (A) and (B) in solution or dispersion in a propellant. In another example, the inhalable form is a nebulizable composition comprising a dispersion of (A) and (B) in an aqueous, organic or aqueous/organic medium.

An aerosol composition suitable for use as the inhalable form of the composition of the invention may comprise the active ingredients in solution or dispersion in a propellant, which may be chosen from any of the propellants known in the art. Suitable such propellants include hydrocarbons such as n-propane, n-butane or isobutane or mixtures of two or more such hydrocarbons, and halogen-substituted hydrocarbons, for example fluorine-substituted methanes, ethanes, propanes, butanes, cyclopropanes or cyclobutanes, particularly 1,1,1,2-tetrafluoroethane (HFA134a) and 1,1,1,2,3,3,3-heptafluoropropane (HFA227), or mixtures of two or more such halogen-substituted hydrocarbons. Where (A) and/or (B) are present in suspension in the propellant, i.e. where present in particulate form dispersed in the propellant, the aerosol composition may also contain a lubricant and a surfactant, which may be chosen from those lubricants and surfactants known in the art. Other suitable aerosol compositions include surfactant-free or substantially surfactant-free aerosol compositions. The aerosol composition may contain up to about 5% by weight, for example 0.002 to 5%, 0.01 to 3%, 0.015 to 2%, 0.1 to 2%, 0.5 to 2% or 0.5 to 1%, by weight of the mixture of (A) and (B), based on the weight of the propellant. Where present, the lubricant and surfactant may be in an amount up to 5% and 0.5% respectively by weight of the aerosol composition. The aerosol composition may also contain a co-solvent such as ethanol in an amount up to 30% by weight of the composition, particularly for administration from a pressurised metered dose inhalation device.

In another embodiment of the invention, the inhalable form is a dry powder, i.e. (A) and (B) are present in a dry powder comprising finely divided (A) and (B) optionally together with a finely divided pharmaceutically acceptable carrier, which is preferably present and may be one or more materials chosen from materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran or mannitol. An especially preferred carrier is lactose, particularly in the form of the monohydrate. The dry powder may be in capsules of gelatin or plastic, or in blisters, for use in a dry powder inhalation device, preferably in dosage units of the mixture of (A) and (B) together with the carrier in amounts to bring the total weight of powder in each capsule to from 5 mg to 50 mg. Alternatively, the dry powder may be contained in a reservoir of a multi-dose dry powder inhalation device.

In the finely divided particulate form of the composition of the invention, (A) and (B) may each have an average particle diameter of up to about 10 μm, for example 0.1 to 5 μm, preferably 1 to 5 μm. In the aerosol composition where (A) and/or (B) are present in particulate form, (A) and/or (B) may have an average particle diameter of up to about 10 μm, for example 0.1 to 5 μm, preferably 1 to 5 μm. The solid carrier, where present, generally has a maximum particle diameter of 300 μm, preferably 212 μm, and conveniently has a mean particle diameter of 40 to 100 μm, preferably 50 to 75 μm. The particle site of the active ingredients (A) and (B), and that of a solid-carrier where present in dry powder compositions, can be reduced to the desired level by conventional methods, for example by grinding in an air-jet mill, ball mill or vibrator mill, microprecipitation, spray-drying, lyophilisation or recrystallisation from supercritical media.

The inhalable pharmaceutical composition of the invention may be administered using an inhalation device suitable for the inhalable form, such devices being well known in the art. Accordingly, the invention also provides a pharmaceutical product comprising a pharmaceutical composition comprising (A) and (B) as hereinbefore described in inhalable form as hereinbefore described in association with one or more inhalation devices. In a further aspect, the invention provides an inhalation device containing a pharmaceutical composition comprising (A) and (B) as hereinbefore described in inhalable form as hereinbefore described.

Where the inhalable form of the composition of the invention is an aerosol composition, the inhalation device may be an aerosol vial provided with a valve adapted to deliver a metered dose, such as 10 to 100 μl, e.g. 25 to 50 μl, of the composition, i.e. a device known as a metered dose inhaler. Suitable such aerosol vials and procedures for containing within them aerosol compositions under pressure are well known to those skilled in the art of inhalation therapy. For example, an aerosol composition may be administered from a coated can, for example as described in EP-A-0642992. Where the inhalable form of the composition of the invention is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer, for example an electronically controlled device such as an AERx (ex Aradigm, US) or a mechanical device such as a RESPIMAT (Boehringer Ingelheim) nebulizer which allows much smaller nebulized volumes, e.g. 10 to 100 μl, than conventional nebulizers. Where the inhalable form of the composition of the invention is the finely divided particulate form, the inhalation device may be, for example, a dry powder inhalation device adapted to deliver dry powder from a capsule or blister containing a dosage unit of the dry powder or a multidose dry powder inhalation (MDPI) device adapted to deliver, for example, 5-25 mg of dry powder per actuation. Suitable such dry powder inhalation devices are well known. For example, a suitable device for delivery of dry powder in encapsulated form is that described in U.S. Pat. No. 3,991,761, while a suitable MDPI device is that described in WO97/20589.

The weight ratio of formoterol, or salt or solvate thereof, to fluticasone propionate may be, in general, from 3:1 to 1:3000, for example from 2:1 to 1:2000, from 1:1 to 1:1000, from 1:2 to 1:500 or from 1:5 to 1:50. More usually, this ratio is from 1:10 to 1 to 1:25, for example from 1:10 to 1:20. Specific examples of this ratio, to the nearest whole number, include 1:10, 1:11, 1:12, 1:13, 1:14, 1:15, 1:16, 1:17, 1:18, 1:19, 1:20, 1:21, 1:22, 1:23, 1:24 and 1:25. The above weight ratios apply particularly where (A) is formoterol fumarate dihydrate. Thus, since the molecular weights of formoterol fumarate dihydrate and fluticasone propionate are 840.9 and 500.6 respectively, the corresponding molar ratios of (A) to (B) may be, in general, from 1.79:1 to 1:5017, for example from 1.2:1 to 1:3345, from 0.6:1 to 1:1672, from 1:3.34 to 1:836 or from 1:8.36 to 1:83.6; more usually from 1:16.7 to 1:41.8, for example from 1:16.7 to 1:33.4; specific examples of the molar ratio being 1:16.7, 1:18.4, 1:20.1, 1:21.7, 1:23.4, 1:25.1, 1:26.8, 1:28.4, 1:30.1, 1:31.8, 1:33.4, 1:35.1, 1:36.8, 1:38.5, 1:40.1, and 1:41.8.

A suitable daily dose of formoterol, or salt or solvate thereof, particularly as formoterol fumarate dihydrate, for inhalation in a composition of the invention may be from 1 to 72 μg, for example from 1 to 60 μg, generally from 3 to 50 μg, preferably from 6 to 48 μg, for instance from 6 to 24 μg. A suitable daily dose of fluticasone propionate for inhalation in a composition of the invention may be from 25 to 3000 μg, for example from 25 to 20000 μg, from 50 to 2000 μg, preferably from 100 to 1000 μg, for instance from 200 to 1000 μg or from 200 to 500 μg. The precise dose used will of course depend on the condition to be treated, the patient and the efficiency of the inhalation device. The formulation of a composition of the invention and its frequency of administration may be chosen accordingly. A suitable unit dose of formoterol component (A), particularly as formoterol fumarate dihydrate, in a composition of the invention may be from 1 to 72 μg, for example from 1 to 60 μg, generally from 3 to 48 μg, preferably from 6 to 36 μg, especially from 12 to 24 μg. A suitable unit dose of fluticasone propionate (B) in a composition of the invention may be from 25 μg to 500 μg, for example from 50 μg to 400 μg, preferably from 100 μg to 300 μg, especially from 150 to 250 μg. These unit doses may suitably be administered once or twice daily in accordance with the suitable daily dose mentioned hereinbefore. For on demand usage, a dosage unit containing 6 μg or 12 μg of (A) and 50 μg or 100 μg of fluticasone propionate (B) is preferred.

In one preferred embodiment of the invention, when the pharmaceutical composition of the invention is a dry powder in a capsule containing a unit dose of (A) and (B), for example for inhalation from a single capsule inhaler, the capsule may suitably contain, where (A) is formoterol fumarate dihydrate, from 3 μg to 36 μg of (A), preferably from 6 μg to 24 μg of (A), especially from 12 μg to 24 μg of (A), and from 25 μg to 500 μg of (B), preferably from 50 μg to 250 μg of (B), especially from 100 to 250 μg of (B), together with a pharmaceutically acceptable carrier as hereinbefore described in an amount to bring the total weight of dry powder per capsule to between 5 mg and 50 mg, for example 5 mg, 10 mg, 15 mg, 20 mg, 25 mg, 30 mg, 35 mg, 40 mg, 45 mg or 30 mg, preferably 20 to 25 mg, especially 25 mg.

In another preferred embodiment of the invention, the pharmaceutical composition of the invention is a dry powder for administration from a reservoir of a multi-dose dry powder inhaler adapted to deliver 3 mg to 25 mg of powder containing a unit dose of (A) and (B) per actuation, for example, where (A) is formoterol fumarate dihydrate, a powder comprising, by weight, 3 to 36 parts, preferably 6 to 24 parts, especially 12 to 24 parts of (A); 25 to 500 parts, preferably 50 to 400 parts, especially 100 to 250 parts of (B); and 2464 to 24972 parts, preferably 4464 to 14972 parts, especially 4464 to 9972 parts of a pharmaceutically acceptable carrier as hereinbefore described.

Treatment of inflammatory or obstructive airways diseases in accordance with the invention may be symptomatic or prophylactic treatment. Inflammatory or obstructive airways diseases to which the present invention is applicable include asthma of whatever type or genesis including both intrinsic (non-allergic) asthma and extrinsic (allergic) asthma. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to ‘morning dipping’. ‘Morning dipping’ is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include acute lung injury (ALI), acute respiratory distress syndrome (ARDS), chronic obstructive pulmonary, airways or lung disease (COPD, COAD or COLD), including chronic bronchitis and emphysema, bronchiectasis and exacerbation of airways hyperreactivity consequent to other drug therapy, in particular other inhaled drug therapy. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

The invention is illustrated by the following Examples, in which parts are by weight unless stated otherwise.

### EXAMPLE 1

### Aerosol Composition for Metered Dose Inhaler

| Ingredient | % by weight |
|---|---|
| Formoterol fumarate dihydrate | 0.012 |
| Fluticasone propionate | 0.250 |
| Ethanol (absolute) | 2.500 |
| HFA 227 | 60.768 |
| HFA134a | 36.470 |

### EXAMPLE 2

### Dry Powder

| Ingredient | % by weight |
|---|---|
| Formoterol fumarate dihydrate | 0.048 |
| Fluticasone propionate | 1.000 |
| Lactose monohydrate | 98.952 |

### EXAMPLE 3

A dry powder suitable for delivery from the reservoir of the multidose inhaler described in WO97/20589 is prepared by mixing 12 parts of formoterol fumarate dihydrate which has been ground to a mean particle diameter of 1-5 μm in an air-jet mill, 250 parts of fluticasone propionate which has been similarly ground to a mean particle diameter of 1-5 μm and 4738 parts of lactose monohydrate having a particle diameter below 212 μm.

### Examples 4-92

Example 3 is repeated, but using the amounts of the ingredients shown in the table below in place of the amounts used in that Example

| | Formoterol | | |
|---|---|---|---|
| | Fumarate | | |
| | Dihydrate | Fluticasone Propionate | Lactose Monohydrate |
| Example | (Parts) | (Parts) | (Parts) |
| 4 | 12 | 50 | 4938 |
| 5 | 12 | 100 | 4888 |
| 6 | 12 | 150 | 4838 |
| 7 | 12 | 200 | 4788 |
| 8 | 6 | 50 | 4944 |
| 9 | 6 | 100 | 4894 |
| 10 | 6 | 150 | 4844 |
| 11 | 6 | 200 | 4794 |
| 12 | 6 | 250 | 4744 |
| 13 | 18 | 50 | 4932 |
| 14 | 18 | 100 | 4882 |
| 15 | 18 | 150 | 4832 |
| 16 | 18 | 200 | 4782 |
| 17 | 18 | 250 | 4732 |
| 18 | 24 | 50 | 4926 |
| 19 | 24 | 100 | 4876 |
| 20 | 24 | 150 | 4826 |
| 21 | 24 | 200 | 4776 |
| 22 | 24 | 250 | 4726 |
| 23 | 30 | 50 | 4920 |
| 24 | 30 | 100 | 4870 |
| 25 | 30 | 150 | 4820 |
| 26 | 30 | 200 | 4770 |
| 27 | 30 | 250 | 4720 |
| 28 | 36 | 50 | 4914 |
| 29 | 36 | 100 | 4864 |
| 30 | 36 | 150 | 4814 |
| 31 | 36 | 200 | 4764 |
| 32 | 36 | 250 | 4714 |
| 33 | 6 | 50 | 9944 |
| 34 | 6 | 100 | 9894 |
| 35 | 6 | 150 | 9844 |
| 36 | 6 | 200 | 9794 |
| 37 | 6 | 250 | 9744 |
| 38 | 12 | 50 | 9938 |
| 39 | 12 | 100 | 9888 |
| 40 | 12 | 150 | 9838 |
| 41 | 12 | 200 | 9788 |
| 42 | 12 | 250 | 9738 |
| 43 | 18 | 50 | 9932 |
| 44 | 18 | 100 | 9882 |
| 45 | 18 | 150 | 9832 |
| 46 | 18 | 200 | 9782 |
| 47 | 18 | 250 | 9732 |
| 48 | 24 | 50 | 9926 |
| 49 | 24 | 100 | 9876 |
| 50 | 24 | 150 | 9826 |
| 51 | 24 | 200 | 9776 |
| 52 | 24 | 250 | 9726 |
| 53 | 30 | 50 | 9920 |
| 54 | 30 | 100 | 9870 |
| 55 | 30 | 150 | 9820 |
| 56 | 30 | 200 | 9770 |
| 57 | 30 | 250 | 9720 |
| 58 | 36 | 50 | 9914 |
| 59 | 36 | 100 | 9864 |
| 60 | 36 | 150 | 9814 |
| 61 | 36 | 200 | 9764 |
| 62 | 36 | 250 | 9714 |
| 63 | 6 | 50 | 14944 |
| 64 | 6 | 100 | 14894 |
| 65 | 6 | 150 | 14844 |
| 66 | 6 | 200 | 14794 |
| 67 | 6 | 250 | 14744 |
| 68 | 12 | 50 | 14938 |
| 69 | 12 | 100 | 14888 |
| 70 | 12 | 150 | 14838 |
| 71 | 12 | 200 | 14788 |
| 72 | 12 | 250 | 14738 |
| 73 | 18 | 50 | 14932 |
| 74 | 18 | 100 | 14882 |
| 75 | 18 | 150 | 14832 |
| 76 | 18 | 200 | 14782 |
| 77 | 18 | 250 | 14732 |
| 78 | 24 | 50 | 14926 |
| 79 | 24 | 100 | 14876 |
| 80 | 24 | 150 | 14826 |
| 81 | 24 | 200 | 14776 |
| 82 | 24 | 250 | 14726 |
| 83 | 30 | 50 | 14920 |
| 84 | 30 | 100 | 14870 |
| 85 | 30 | 150 | 14820 |
| 86 | 30 | 200 | 14770 |
| 87 | 30 | 250 | 14720 |
| 88 | 36 | 50 | 14914 |
| 89 | 36 | 100 | 14864 |
| 90 | 36 | 150 | 14814 |
| 91 | 36 | 200 | 14764 |
| 92 | 36 | 250 | 14714 |

### EXAMPLE 93

Gelatin capsules suitable for use in a capsule inhaler such as that described in U.S. Pat. No. 3,991,761 are prepared, each capsule containing a dry powder obtained by mixing 12 μg of formoterol fumarate dihydrate which has been ground to a mean particle diameter of 1 to 5 μm in an air jet mill, 250 μg of fluticasone propionate which has been similarly ground to a mean particle diameter of 1 to 5 μm and 24738 μg of lactose monohydrate having a particle diameter below 212 μm.

### Examples 94-152

Example 93 is repeated, but using the amounts of the ingredients shown in the table below in place of the amounts used in that Example:

| | Formoterol | | |
|---|---|---|---|
| | Fumarate | | |
| | Dihydrate | Fluticasone Propionate | Lactose Monohydrate |
| Example | (Parts) | (Parts) | (Parts) |
| 94 | 12 | 50 | 24938 |
| 95 | 12 | 100 | 24888 |
| 96 | 12 | 150 | 24838 |
| 97 | 12 | 200 | 24788 |
| 98 | 6 | 50 | 24944 |
| 99 | 6 | 100 | 24894 |
| 100 | 6 | 150 | 24844 |
| 101 | 6 | 200 | 24794 |
| 102 | 6 | 250 | 24744 |
| 103 | 18 | 50 | 24932 |
| 104 | 18 | 100 | 24882 |
| 105 | 18 | 150 | 24832 |
| 106 | 18 | 200 | 24782 |
| 107 | 18 | 250 | 24732 |
| 108 | 24 | 50 | 24926 |
| 109 | 24 | 100 | 24876 |
| 110 | 24 | 150 | 24826 |
| 111 | 24 | 200 | 24776 |
| 112 | 24 | 250 | 24726 |
| 113 | 30 | 50 | 24920 |
| 114 | 30 | 100 | 24870 |
| 115 | 30 | 150 | 24820 |
| 116 | 30 | 200 | 24770 |
| 117 | 30 | 250 | 24720 |
| 118 | 36 | 50 | 24914 |
| 119 | 36 | 100 | 24864 |
| 120 | 36 | 150 | 24814 |
| 121 | 36 | 200 | 24764 |
| 122 | 36 | 250 | 24714 |
| 123 | 6 | 50 | 19944 |
| 124 | 6 | 100 | 19894 |
| 125 | 6 | 150 | 19844 |
| 126 | 6 | 200 | 19794 |
| 127 | 6 | 250 | 19744 |
| 128 | 12 | 50 | 19938 |
| 129 | 12 | 100 | 19888 |
| 130 | 12 | 150 | 19838 |
| 131 | 12 | 200 | 19788 |
| 132 | 12 | 250 | 19738 |
| 133 | 18 | 50 | 19932 |
| 134 | 18 | 100 | 19882 |
| 135 | 18 | 150 | 19832 |
| 136 | 18 | 200 | 19782 |
| 137 | 18 | 250 | 19732 |
| 138 | 24 | 50 | 19926 |
| 139 | 24 | 100 | 19876 |
| 140 | 24 | 150 | 19826 |
| 141 | 24 | 200 | 19776 |
| 142 | 24 | 250 | 19726 |
| 143 | 30 | 50 | 19920 |
| 144 | 30 | 100 | 19870 |
| 145 | 30 | 150 | 19820 |
| 146 | 30 | 200 | 19770 |
| 147 | 30 | 250 | 19720 |
| 148 | 36 | 50 | 19914 |
| 149 | 36 | 100 | 19864 |
| 150 | 36 | 150 | 19814 |
| 151 | 36 | 200 | 19764 |
| 152 | 36 | 250 | 19714 |

### Examples 153-176

Example 3 is repeated, but using the amounts of the ingredients shown in the table below in place of the amounts used in that Example:

| | Formoterol | | |
|---|---|---|---|
| | Fumarate | | |
| | Dihydrate | Fluticasone Propionate | Lactose Monohydrate |
| Example | (Parts) | (Parts) | (Parts) |
| 153 | 6 | 25 | 2969 |
| 154 | 6 | 50 | 2944 |
| 155 | 6 | 100 | 2894 |
| 156 | 6 | 150 | 2844 |
| 157 | 6 | 200 | 2794 |
| 158 | 6 | 250 | 2744 |
| 159 | 12 | 25 | 2963 |
| 160 | 12 | 50 | 2938 |
| 161 | 12 | 100 | 2888 |
| 162 | 12 | 150 | 2838 |
| 163 | 12 | 200 | 2788 |
| 164 | 12 | 250 | 2738 |
| 165 | 12 | 300 | 2638 |
| 166 | 12 | 350 | 2588 |
| 167 | 12 | 400 | 2538 |
| 168 | 24 | 25 | 2951 |
| 169 | 24 | 50 | 2926 |
| 170 | 24 | 100 | 2876 |
| 171 | 24 | 150 | 2826 |
| 172 | 24 | 200 | 2776 |
| 173 | 24 | 250 | 2726 |
| 174 | 24 | 300 | 2676 |
| 175 | 24 | 350 | 2626 |
| 176 | 24 | 400 | 2576 |

### Examples 177-216

Example 93 is repeated, but using the amounts of the ingredients shown in the table below in place of the amounts used in that Example:

| | Formoterol Fumarate | Fluticasone | Lactose |
|---|---|---|---|
| Example | Dihydrate (μg) | Propionate (μg) | Monohydrate (μg) |
| 177 | 6 | 25 | 14969 |
| 178 | 6 | 50 | 14944 |
| 179 | 6 | 100 | 14894 |
| 180 | 6 | 150 | 14844 |
| 181 | 6 | 200 | 14794 |
| 182 | 6 | 250 | 14744 |
| 183 | 6 | 300 | 14694 |
| 184 | 6 | 350 | 14644 |
| 185 | 6 | 400 | 14594 |
| 186 | 12 | 25 | 14963 |
| 187 | 12 | 50 | 14938 |
| 188 | 12 | 100 | 14888 |
| 189 | 12 | 150 | 14838 |
| 190 | 12 | 200 | 14788 |
| 191 | 12 | 250 | 14738 |
| 192 | 12 | 300 | 14688 |
| 193 | 12 | 350 | 14638 |
| 194 | 12 | 400 | 14588 |
| 195 | 12 | 500 | 14488 |
| 196 | 24 | 25 | 14951 |
| 197 | 24 | 50 | 14926 |
| 198 | 24 | 100 | 14876 |
| 199 | 24 | 150 | 14826 |
| 200 | 24 | 200 | 13876 |
| 201 | 24 | 250 | 13826 |
| 202 | 24 | 300 | 13776 |
| 203 | 6 | 25 | 9969 |
| 204 | 6 | 50 | 9944 |
| 205 | 6 | 100 | 9894 |
| 206 | 6 | 150 | 9844 |
| 207 | 6 | 200 | 9794 |
| 208 | 6 | 250 | 9744 |
| 209 | 6 | 300 | 9694 |
| 210 | 12 | 25 | 9963 |
| 211 | 12 | 50 | 9938 |
| 212 | 12 | 100 | 9888 |
| 213 | 12 | 150 | 9838 |
| 214 | 12 | 200 | 9788 |
| 215 | 12 | 250 | 9738 |
| 216 | 12 | 300 | 9688 |

What is claimed is:

1. A pharmaceutical composition comprising (A) formoterol or a pharmaceutically acceptable salt thereof or a solvate of formoterol or said salt and (B) fluticasone propionate.

2. A composition according to claim 1 comprising a mixture of effective amounts of (A) and (B) together with a pharmaceutically acceptable carrier.

3. A composition according to claim 1, in which (A) is formoterol fumarate.

4. A composition according to claim 3, in which formoterol fumarate is in the form of the dihydrate thereof.

5. A composition according to claim 1, which is in inhalable form.

6. A composition according to claim 4, which is in inhalable form.

7. A composition according to claim 5, which is an aerosol comprising a mixture of (A) and (B) in solution or dispersion in a propellant.

8. A composition according to claim 7, in which (A) and (B) are in suspension in said propellant, which is a halogen-substituted hydrocarbon.

9. A composition according to claim 8, in which (A) and (B), or each of (A) and (B), has an average particle diameter of up to 10 μm.

10. A composition according to claim 5, which is a nebulizable composition comprising a dispersion of (A) and (B) in an aqueous, organic or aqueous/organic medium.

11. A composition according to claim 5, which is a dry powder comprising finely divided (A) and (B) optionally together with a pharmaceutically acceptable carrier in finely divided form.

12. A composition according to claim 11, in which the carrier is present and is a saccharide.

13. A composition according to claim 12, in which the carrier is lactose.

14. A composition according to claim 11 in which (A) or (B), or each of (A) and (B), has an average particle diameter of up to 10 μm.

15. A composition according to claim 1, in which the weight ratio of (A) to (B) is from 3:1 to 1:3000.

16. A composition according to claim 15, in which said ratio is from 1:5 to 1:50.

17. A composition according to claim 15, in which said ratio is from 1:10 to 1:25.

18. A composition according to claim 1, which is a dry powder in a capsule, the capsule containing from 3 to 36 μg of (A) as formoterol fumarate dihydrate, from 25 to 500 μg of (B) and a pharmaceutically acceptable carrier in an amount to bring the total weight of dry powder to between 5 mg and 50 mg.

19. A composition according to claim 1 which is a dry powder comprising, by weight, 3 to 36 parts of (A) as formoterol fumarate dihydrate, 25 to 500 parts of (B) and 4464 to 24972 parts of a pharmaceutically acceptable carrier.

20. A method of treating an inflammatory or obstructive airways disease which comprises administering to a subject in need of such treatment an effective amount of a composition according to claim 1.
